(19) United States
(12) Patent Application Publication
(10) Pub. No.: US 2025/0025065 A1
(43) Pub. Date: Jan. 23, 2025

(54) LOW-COST METHOD FOR THE DIAGNOSIS OF CHRONIC OBSTRUCTIVE PULMONARY DISEASE USING ENSEMBLE ML MODEL

(71) Applicants: Advait Achanta, Hyderabad (IN); Suhaas Devalapally, Hyderabad (IN)

(72) Inventors: Advait Achanta, Hyderabad (IN); Suhaas Devalapally, Hyderabad (IN)

(73) Assignees: Advait Achanta, Hyderabad, TG (IN); Suhaas Devalapally, Hyderabad, TG (IN)

(21) Appl. No.: 18/488,068

(22) Filed: Oct. 17, 2023

(30) Foreign Application Priority Data
Jul. 21, 2023 (IN) 202341049329

(51) Int. Cl. A61B 5/08; A61B 5/00; G16H 50/20

(52) U.S. Cl. CPC A61B 5/082; A61B 5/4842; G16H 50/20; G16H 10/60

(57) ABSTRACT

The present invention is related to a simple, low-cost method for the early diagnosis of COPD in patients using an ensemble ML model residing on cloud or server. A web-based application accessed on a smart phone or a computing device through internet facilitates the entry of patient characteristics such as age, gender, smoking habits, hypertension, pack history, etc. and clinical features such as FEV, MWT and CO levels in the exhaled breath of patients. The patient characteristics is used with the ensemble ML model to predict the severity of COPD disease. The web application also enables sending the results to a receiver via email or text message for further action.

### CROSS-REFERENCE TO RELATED APPLICATIONS

This application claims priority to application No. 202341049329, filed in India on Jul. 21, 2023. The contents of the foregoing application are incorporated herein by reference.

### FIELD OF THE INVENTION

The present invention is related to the diagnosis of Chronic Obstructive Pulmonary Disease (COPD) in a patient using a patient's demographic data and clinical features. The invention uses an ensemble Machine Learning (ML) model to detect COPD condition in an undiagnosed patient based on said patient data.

### BACKGROUND OF THE INVENTION

Chronic obstructive pulmonary disease (COPD) caused by air pollution and smoking is the third leading cause of death according to the World Health Organization causing 3.23 million deaths in 2019. Most people are unaware that they have COPD because symptoms do not necessarily present themselves early and may not be detected until later stages. In India, the number of COPD cases is high due to the large number of smokers in rural areas and also high air pollution caused by vehicular traffic. 32% of global COPD cases occur in India and COPD alone accounts for over 9.5% of the total deaths. Despite the huge and growing burden of COPD in India, over 98% of people living in urban slums and the rural villages have never heard the word COPD. In addition, many people cannot afford the expensive diagnostic tests that are conducted on the patients during diagnosis. Hence, there is a need for a cost-effective method for early detection of COPD.

US20180261330 from Roundglass LLC discloses a computer-implemented method that uses a set of multiple health outcomes and costs for a stakeholder. Each cost corresponds to a respective health outcome. This application discloses a computer implemented method wherein the multiple stakeholder entities are defined along with their health outcomes and costs, and an equation has been developed wherein certain weightage is accorded based on the number of episodes associated with a particular cost. It discloses the assessment of the value of treatment of Chronic Obstructive Pulmonary Disease (COPD) based on this equation. This invention has used BODE score, a multidimensional system widely used to assess COPD treatment, comprising four components-nutritional state (BMI), airflow limitation (Obstruction; FEV1), breathlessness, (MRC Dyspnea scale), and Exercise capacity (6MWD, distance walked in 6 min). It further discloses use of a decision tree model for classification of COPD treatments by value, wherein advanced stage COPD patients who are heavy smokers are projected with least value of treatment, whereas patients in early stages of COPD and who do not smoke are projected with the highest value of treatment. The results illustrate how the value analytic platform can be used to assess value of COPD treatments based solely on patient risk measures, without any measurements of cost or treatment outcomes per se.

CN111613325A from BOE Technology Group Co. discloses a method, device, electronic device and storage medium for predicting the recurrence of COPD. It discloses a method based on a pre-trained COPD prediction model using physical examination data, living environment data, living habits data and other user characteristic data. The model based on decision tree is used to predict the recurrence of COPD in patients in the phase prior to sickness, so that the patients can prevent the disease before the disease worsens. User characteristic data such as body detection data including ventilator, oxygen generator and pulmonary function output data, living environment data (temperature, humidity and PM value), and living habits data (smoking, coughing, and body comfort) are acquired. Based on the user characteristic data, at least three attributes were determined and values were assigned to the attributes to obtain an attribute set which is input into the pre-trained COPD prediction model to obtain the COPD recurrence prediction result of the user. The COPD prediction model is trained using a classification and regression tree (CART) algorithm based on a decision tree.

CN113257416A from Zhejiang University (ZJU) discloses a method for individualized management and optimization of COPD patients based on deep learning, comprising the steps of obtaining current symptom indicators of COPD patients, recommending an intervention strategy based on the current symptom index, generating an input vector based on the current symptom index and recommended intervention strategy, inputting the input vector to the trained COPD virtual intervention environment model to obtain an output vector, wherein, the output vector represents the symptom index of the COPD patient after the intervention strategy, calculating the improvement value based on the current symptom index and the symptom index after the intervention, and updating the evaluation standard of the current intervention strategy combination.

Md. Arshad Ejazi et al. reported assessment of the severity of COPD disease and treatment response by measuring exhaled carbon monoxide (eCO) as a biomarker. COPD was diagnosed based on clinical examinations, Spirometry and eCO values.

Catarina Duarte Santos et al. conducted a study to understand the feasibility of exercise-field tests to identify patients who desaturate (SpO₂<90%) during physical activities. This study compared the six-minute walk test (6MWT) and daily-life telemonitoring.

Alda Marques et al. from the University of Aveiro (ESSUA) conducted a study to identify and describe profiles and corresponding treatable traits in people with COPD based on simple and meaningful clinical measures collected with minimal resources. The authors developed and validated a decision tree to quickly identify the profile of each person to assess the stability of the profiles during a six-months period. COPD Assessment Test (CAT), age, FEV were measured for the assessment.

Although the aforementioned prior art teaches the use of different techniques such as Machine Learning, Decision Tree (DT) etc. for determining an early prediction of COPD, none of them have considered the use of demographic data of patients and clinical features including CO in the exhaled breath that have been considered in the current invention. Also, use of an ensemble model has not been cited in any of the references cited above.

### OBJECT OF THE INVENTION

The primary object of the present invention is to provide a simple, low-cost method for the early diagnosis of COPD.

Another object of the present invention is to provide a simple low-cost method for the detection of COPD in patients using a combination of patient characteristics and clinical features of patients.

Yet another object of the present invention is to use patient characteristics such as patient's age, gender, hypertension, smoking, habits and pack history, for the diagnosis of COPD.

Still yet another object of the present invention is to use clinical features of patients such as Forced Expiratory Volume (FEV) which measures volume of exhaled air during a forced breath, and a Six Minute Walk Test which assesses functional exercise capacity during walking, for the diagnosis of COPD.

Further yet another object of the present invention is to provide a low-cost electronic handheld carbon monoxide (CO) analyzer that can be used for measuring the CO level in exhaled breath of COPD patients.

Another object of the present invention is to process patient characteristics and patient clinical features using Machine Learning (ML) techniques for the diagnosis of COPD.

Another object of the present invention is to use ensemble methods in ML to provide an accurate method for COPD detection.

A further object of the present invention is to have a web-based application on a smart phone or any computing device provided with an ensemble ML model, for use in the diagnosis of COPD.

Another object of the present invention is to provide a means to display the severity of COPD disease on said smart phone for taking further action.

Another object of the present invention is to enable remote monitoring of COPD disease through transfer of diagnosed COPD results from said smart phone or computing device to any third party receiver.

### SUMMARY OF THE INVENTION

The present invention provides a simple, low-cost method and device for the detection of Chronic Obstructive Pulmonary Disease (COPD) in patients with respiratory conditions using a combination of demographic data and clinical features of the patient. Said method comprises feeding acquired data into a web-based application on a smart phone or any computing device from where it is sent to the cloud where an ensemble ML model resides. The ensemble model assesses the severity of the COPD disease and the results are displayed on said smart phone or computing device. The said invention also provides a simple, low-cost, electronic hand-held device for measuring the exhaled CO in a patient's breath.

### BRIEF DESCRIPTION OF THE DRAWINGS

The summary of the present invention, as well as the detailed description, is better understood when read in conjunction with the accompanying drawings that illustrate one or more possible embodiments of the present invention, of which:

FIG. 1 illustrates the primary rules generated by the Decision Tree model after training the patient's demographic data and clinical features;

FIG. 2 is a graphical representation that illustrates the patient data that is classified after the application of each of the Decision Tree rules illustrated in FIG. 1;

FIG. 3 illustrates the ensemble ML method used for testing the patient data;

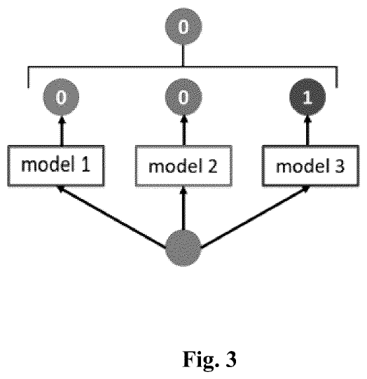

FIG. 4 illustrates the web-based application on a smart phone or any computing device displaying the values obtained from the testing of COPD patients; and

FIG. 5 illustrates an electronic, hand held, low-cost CO analyzer.

### DETAILED DESCRIPTION OF THE INVENTION

COPD is a progressive lung disease characterized by obstruction of airflow that leads to difficulty in breathing. COPD affects millions of people worldwide and is a significant cause of morbidity and mortality. Accurate diagnosis and classification of COPD patients are crucial for effective treatment and management of the disease. The diagnosis is also based on findings from the X-ray and CT scans of patient's lungs.

In rural areas of India or any such developing nation where there are Primary Health Centres without an X-ray or CT machine, said invention will enable the healthcare technician at the Primary Health Centre decide on whether the patient is to be sent to a hospital in the nearest town or city for further investigations related to COPD.

In recent years, ML has become a powerful tool that is being used in different technology domains. It is devoted to creating algorithms and models that allow software applications to “learn” and perform accurate predictions upon the existing plethora of data. Thus, through ML, computers are capable of automatically improving their functionalities based on experience. However, computers have to utilize data derived from the real world so that they can “learn” from them and provide the required predictions. To accomplish such a task, the applied ML techniques are usually separated into two categories, namely, supervised and unsupervised ML techniques. A labeled sample of training data is utilized first using Decision Tree, to estimate or map the input data to obtain the intended output. Random Forest is used for the assessment of functional capacity when no labeled data is supplied, and hence no specified intended result is obtained. However, the goal is to discover patterns, structures, or relationships in unlabeled data without any explicit guidance or labeled examples. In every case, the ultimate goal remains the same-generation of a ML model that can be exploited for classification, prediction, or any other related task. When the model is generated, it is imperative that it is evaluated. In this context, the model's performance is assessed based on specific metrics such as accuracy, specificity, recall, precision F1-score, and confusion matrix among others.

In general, ML tools have grown in popularity in the healthcare area during the last few decades, where various ML algorithms including BNB, KNN, Decision Tree (DT), Logistic Regression (LR), Random Forest (RF), Neural Networks (NN), and Stochastic Gradient Descent (SGD) among others, have been widely applied, aiming to detect key features of patients' conditions, health risks, as well as disease progression after treatment, exploiting information that is derived from various complex medical datasets.

The object of the current invention is to provide a simple, low-cost method that uses an ensemble ML model that can accurately classify patients as COPD patients or non-COPD patients based on the given criteria. The criteria considered for the ML model includes patient characteristics such as age, gender, hypertension condition, smoking history, and number of packs of cigarettes smoked (pack history) as well as patient's clinical features such as CO in exhaled breath, results from tests on FEV, and Six-Minute Walking test (MWT).

The ML model chosen for the present invention is an ensemble ML model that combines Decision Tree (DT), Random Forest, and Gradient Boosting. Ensemble modeling is a technique in machine learning where multiple models are combined to obtain better predictive performance instead of using a single model. It is based on the principle of aggregating the predictions from multiple models which can often lead to improved accuracy, robustness, and generalization ability. Decision Tree is analogous to the flowchart structure, wherein each internal node represents a condition for an attribute, each branch represents the condition's outcome, each leaf node represents the class label, and the final choice is made after computing all the attributes. In essence, it employs a decision tree to move from observations of an item (represented by branches) to inferences about the target value of the item (represented by leaves). Random Forest combines the output of multiple decision trees to reach a single result. It handles both classification and regression problems. Gradient Boosting is a supervised learning algorithm that is built by combining Decision Tree with a technique called boosting.

In the present invention, patient characteristics such as age, gender, hypertension, smoking, and pack history of patients with respiratory conditions are collected by the healthcare technician. The technician examines the patient and measures the FEV value, MWT, and percentage of CO in the patient's exhaled breath. The FEV values are generally measured using spirometry. Results from the Six-MWT are also collected by the health care technician.

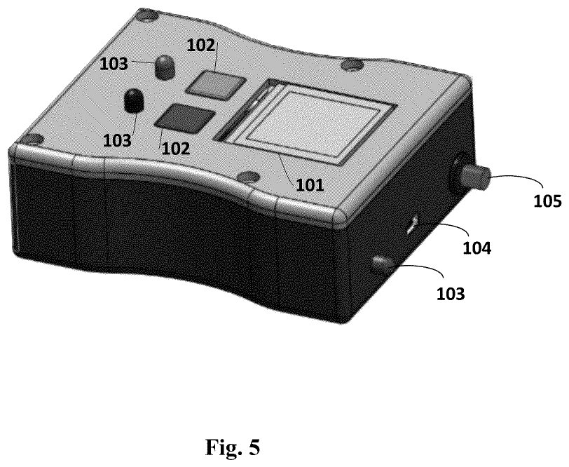

FIG. 5 shows a low-cost electronic handheld CO analyzer. For enabling low-cost CO measurement, the inventors of the present invention developed a low-cost breath analyzer that measures the % CO in the exhaled breath of a patient. The 3D printed hand-held CO analyzer has a 1.5-inch OLED display (101) which displays results, two buttons (102) which are to be selected depending on whether the patient breathing into the device is a smoker or a non-smoker, three LEDs (103) which are used as indicators to show that the device is working, and to signal when the patient is required to breathe in or stop, and the Spec CO Sensor and an ON-OFF switch (105). The CO analyzer comprises a Spec CO Sensor which is calibrated using a CO Gas Detector. An app was developed using Tkinter (a python library) on Visual Studio Editor. The Arduino Nano was connected to Raspberry Pi 4 through a Mini-B USB connector (104). All the data was transferred in a specified string format from the Nano to Raspberry Pi. After receiving the data, the Raspberry Pi is split to match each type of data for the app to display. The app displays the environment CO level, the highest carbon monoxide level in the breath as well as the average carbon monoxide level in the breath.

In order to obtain a more precise diagnosis of COPD, a combination of three different ML algorithms were applied to the patient data instead of just one ML algorithm. The ML algorithms applied were Decision Tree, Random Forest, and Gradient Boosting. While Decision Tree considers rules based on a single tree for classification of data, Random Forest uses multiple trees for classification of data. Gradient Boosting applies gradients for predicting data. Initially, said models were trained individually with training data following which a combination of three models was used for testing.

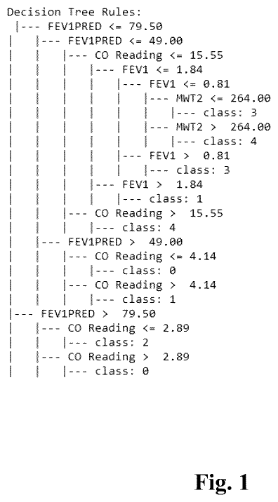

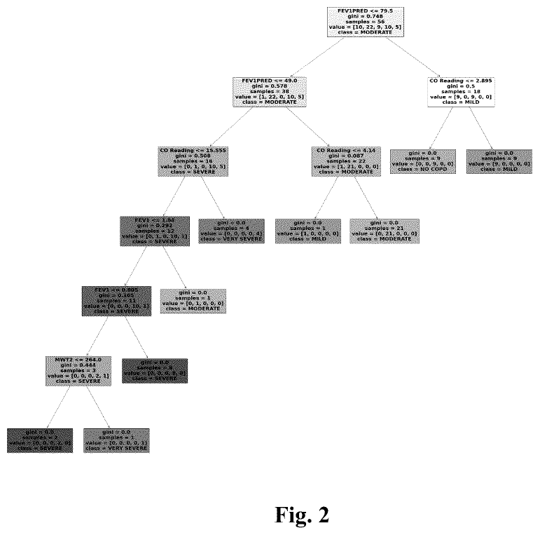

With the use of Decision Tree for training, the Decision rules for classification were obtained as shown in FIG. 1, wherein FEV and CO values have been considered. Prediction of severity of COPD disease based on the rules of Decision Tree model has been presented in FIG. 2. The severity has been identified as “No COPD”, “Mild”, “Moderate”, “Severe”, and “Very Severe”.

While predicting using ensemble ML model, a single data point was passed to all the three models, namely, Decision Tree, Random Forest and Gradient Boosting, and the output from each of the models was collected. A voting method was used to determine the severity of the disease in the output. In case the output regarding the severity of the disease from two or three models was identical, the output was considered to be as obtained. In case the output models generated non-identical values of disease severity, then the least value among the three output values was considered. For example, in case the outputs generated were ‘Mild’, ‘Moderate’, and ‘Severe’ from different models for the same dataset, then the output considered was ‘Mild’.

Prior to applying the data to ensemble ML model consisting of Decision Tree, Random Forest, and Gradient Boosting, the dataset was preprocessed. This comprised handling missing values, and label encoding of categorical variables. Missing values were addressed by taking the average of the patient data pertaining to a parameter. In the case of label encoding of categorical values, ‘No COPD’, ‘Mild’, ‘Moderate’, ‘Severe’ and ‘Very severe’ were encoded as zero, one, two, three, and four, respectively.

The dataset was split into training and testing sets to evaluate the performance of the model. 80% of data was used for training, and 20% of the data was used for testing the accuracy of the model. The training set was used to train the ensemble model, while the testing set was used to evaluate its performance. Accuracy, precision, recall, and F1-score obtained from the ensemble model is given below.
Precision: 0.95
Recall: 0.93
F1 Score: 0.92
Accuracy: 0.93

The trained ensemble ML model was uploaded on the cloud.

One of the key advantages of including Decision Tree in ensemble ML model, is its ability to provide insight into the importance of features. Thus, the decision paths and splits within the tree were analyzed, and the most important features that make significant contribution to the classification were identified. This helps pinpoint the factors that influence the presence or absence of COPD.

Decision Tree classifiers have hyperparameters that control the tree's growth and prevent overfitting. Said hyperparameters include the maximum depth of the tree, minimum samples required for a split, and minimum samples required at a leaf node. Generally, five levels are considered throughout the length from root node to child node. The main advantage of decision tree classification is its interpretability. The decision tree model provides a clear and understandable representation of the classification rules, which can be easily interpreted by clinicians and healthcare professionals. This interpretability can aid in clinical decision-making, as it allows medical experts to understand the reasoning behind the classification and helps make informed treatment decisions.

Decision tree classification is a valuable tool for the classification of COPD data sets. By leveraging the features of the dataset, decision tree models can accurately classify patients as COPD or non-COPD, providing valuable insights for diagnosis and treatment planning. The interpretability of Decision trees further enhances their utility in the medical field, allowing for transparent and explainable decision-making. However, by applying the ensemble method, the accuracy of prediction of COPD is reinforced compared to the COPD prediction obtained by using only Decision Tree method.

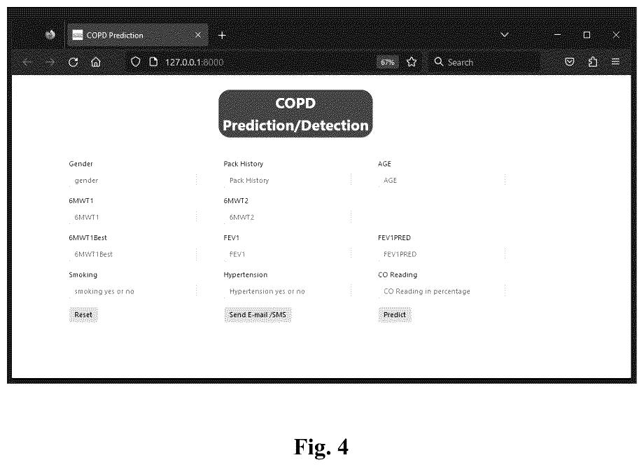

A web-based application was created for feeding the data obtained from the patients. The web-based application was accessed using a smart phone connected to the internet. Following the acquisition of data from patients, the data is fed into said web-based application on the smart phone. A low-cost feature phone having access to internet can also be used in place of smart phones. The display of the web application on the smart phone or computing device as shown in FIG. 4 shows three buttons namely Reset, Send E-mail/SMS, and Predict. RESET button is used to clear the pre-existing values under set fields prior to entering data from a new patient. On feeding the patient data into the web page, the PREDICT button is clicked, and the values are sent to the cloud or server where they are pre-processed and predicted using ensemble model. The diagnosis is then sent back to the computing device or smart phone. The diagnosis about a patient suffering from COPD or not, and if so, the severity of the disease, is displayed on the screen of the computing device or smart phone. Next, based on the results indicating the severity of COPD, the healthcare technician can click on the Send E-mail/SMS button on the display window of the computing device or smart phone, to forward the displayed diagnosis via E-mail, SMS or text message, to the concerned patient and also the healthcare professional at a hospital with facilities such as X-Ray, CT etc. to decide on further evaluation or the course of treatment. On clicking the RESET button, the data is erased and the smart phone or computing device is ready for receiving the next patient's data.

According to various embodiments of the present invention, the objects of the present invention are achieved through an ensemble ML model residing on a cloud or server. Patient characteristics such as age, gender, smoking habits, hypertension, pack history and also clinical features such as FEV, MWT, and CO in exhaled breath are entered into a web application which is accessed through a computing device or smart phone. Said computing device or smart phone displays Reset, Send E-mail/SMS and Predict buttons on the screen. When the healthcare technician feeds the patient data into the computing device or smart phone and clicks the Predict button, the data is pre-processed and predicted by the ensemble ML program residing on the cloud, and the presence or absence of COPD and its severity is displayed on the computing device or smart phone. On clicking the send E-mail/SMS button, the data is forwarded to the patient and a healthcare professional at a hospital with facilities for follow-up action.

It is to be understood, however, that the present invention would not be limited by any means to the components, arrangements, and materials that are not specifically described, and any change to the components, variations, and modifications can be made without departing from the spirit and scope described in the present invention.

1. A method for the diagnosis of chronic obstructive pulmonary disease (COPD) in patients with respiratory conditions comprising:
obtaining patient characteristics and clinical features;
feeding said patient characteristics and clinical features into a web application on a computing device;
using an ensemble machine learning (ML) model residing on the cloud; and
displaying the severity of COPD on said computing device,
wherein said ensemble ML model is a combination of Decision Tree, Random Forest and Gradient Boosting models.

2. The method for the diagnosis of chronic obstructive pulmonary disease (COPD) according to claim 1, wherein said patient characteristics comprise information about the age, gender, hypertension, smoking habits, and pack history of said patient.

3. The method for the diagnosis of chronic obstructive pulmonary disease (COPD) according to claim 1, wherein said clinical features comprise Force Expiratory Volume (FEV), six-minute walking test (MWT), and percentage of carbon monoxide (CO) in exhaled breath of a patient.

4. The method for the diagnosis of chronic obstructive pulmonary disease (COPD) according to claim 3, wherein carbon monoxide is measured using a handheld CO analyzer.

5. The method for the diagnosis of chronic obstructive pulmonary disease (COPD) according to claim 1, wherein said severity of COPD is identified as “No COPD”, “mild”, “moderate”, “severe”, or “very severe”.

6. The method for the diagnosis of chronic obstructive pulmonary disease (COPD) according to claim 1, wherein the diagnosis displayed on the computing device is transmitted to a receiver via an email or a text message.
